(19) United States
(12) Patent Application Publication
(10) Pub. No.: US 2022/0296598 A1
(43) Pub. Date: Sep. 22, 2022

(54) Poziotinib Combination with VEGFR2 Inhibitors and Methods of Use Thereof

(71) Applicant: Spectrum Pharmaceuticals, Inc., Irvine, CA (US)

(72) Inventor: Guru Reddy, Irvine, CA (US)

(73) Assignee: Spectrum Pharmaceuticals, Inc., Irvine, CA (US)

(21) Appl. No.: 17/637,304

(22) PCT Filed: Aug. 21, 2020
(86) PCT No.: PCT/US20/47489

§ 371 Date: Feb. 22, 2022

Related U.S. Application Data
(60) Provisional application No. 62/891,021, filed on Aug. 23, 2019.

(51) Int. Cl. A61K 31/517; C07K 16/28; A61K 39/395; C12Q 1/6886

(52) U.S. Cl. CPC A61K 31/517; C07K 16/2863; A61K 39/3955; C12Q 1/6886; C07K 2317/21; C12Q 2600/156

(57) ABSTRACT

A drug combination containing poziotinib or a pharmaceutically acceptable salt thereof and a VEGFR2 inhibitor and methods of using the combination for treating cancer in a subject in need thereof. The drug combination demonstrates synergistic effect in treating cancers having one or more EGFR or HER2 mutations.

### TECHNICAL FIELD

Disclosed herein is a drug combination containing poziotinib or a pharmaceutically acceptable salt thereof and a VEGFR inhibitor and methods of using the combination for treating cancer in a subject in need thereof.

### BACKGROUND

In recent years, expression of resistance in EGFR target therapy has been reported to decrease response time of the drug used. It has been reported that non-small-cell lung carcinoma (NSCLC) patients having EGFR activating mutations treated with gefitinib or erlotinib are resistant to the drug after about 8 to 16 months of treatment, and about 60% of the patients are observed to be resistant due to the EGFR T790M mutation (Helena A. Yu et al., Clin. Cancer Res. 19(8), 2240, 2013). In addition, in cases of HER2 positive metastatic breast cancer patients treated with the antibody drug, trastuzumab, 66% to 88% of the patients are known to exhibit de novo resistance or acquired resistance due to various mechanisms (Alice Chung et al., Clin. Breast Cancer 13(4), 223, 2013). In this regard, the development of an EGFR-targeting therapeutic agent is limited since its efficacy cannot be maintained for an extended period of time due to generation of primary and secondary resistance, despite the fact that the EGFR targeting therapeutic agent has considerable effect on the treatment of solid cancers with HER2 overexpression or mutation. Thus, improved and alternative materials for dental and medical applications are urgently needed.

### SUMMARY

This patent document provides methods for reducing the risk of, or preventing, tumors from increasing in size or from reaching a metastatic state and therefore serving the objective to halt the progression or advancement of cancer.

An aspect of the patent document provides drug combination for treating a neoplasm in a subject. The combination includes poziotinib or a pharmaceutically acceptable salt thereof and a vascular endothelial growth factor receptor (VEGFR) inhibitor. In some embodiments, the inhibitor is a VEGFR2 inhibitor. In some embodiments, the VEGFR2 inhibitor is Ramucirumab.

An aspect of the patent document provides a kit for treating a neoplasm in a subject. The kit includes poziotinib or a pharmaceutically acceptable salt thereof and a vascular endothelial growth factor receptor 2 (VEGFR2) inhibitor. In some embodiments, the VEGFR2 inhibitor is Ramucirumab. The kit may also include an educational information and direction of use.

Another aspect of this document provides a method of treating a neoplasm in a subject, comprising administering a drug combination described herein to a subject in need thereof.

In some embodiments, the neoplasm is selected from the group consisting of non-small cell lung cancer, breast cancer, stomach cancer, colon cancer, pancreatic cancer, prostate cancer, myeloma, head and neck cancer, ovarian cancer, esophageal cancer, and metastatic cell carcinoma. In some embodiments, the VEGFR2 inhibitor in the drug combination is Ramucirumab.

In some embodiments, the subject expresses a mutation at exon 18, exon 19, exon 20, or exon 21. In some embodiments, the method includes determining the subject as having one or more HER2 exon 20 mutations. In some embodiments, the method includes determining the subject as having one or more EGFR exon 20 mutations selected from the group consisting of A775insV G776C, A775insYVMA, G776C V777insC, G776del insVV, G776del insVC, P780insGSP, Y772dupYVMA, V773M, G776delinsLC, V777L, V777insCG, G778dupGSP, P780insGSP, L786V. In some embodiments, the method includes determining the subject as having one or more EGFR exon 20 mutations selected from the group consisting of A763insFQEA, A767insASV, S768dupSVD, V769insASV, D770insSVD, D770insNPG, H773insNPH, N771del insGY, N771del insFH, N771dupNPH, A767insTLA, S768I, V769L, V769insGSV, D770del insGY, D770insG, D770insY H773Y, N771insHH, P772insDNP, H773insAH, H773insH, V774insHV, S784F, R776C, V774M, V769M, G796D, S784F, C775Y, S811F, T790M, V774A, and D770A.

In some embodiments, the method includes determining the subject as having one or more HER2 exon 21 mutations selected from the group consisting of a point mutation, insertion, and deletion of 1-18 nucleotides between amino acids 832-883. In some embodiments, the method includes determining the subject as having one or more HER2 exon 21 mutations are at one or more residues selected from the group consisting of V842, R868, and L869.

In some embodiments, the neoplasm is non-small cell lung carcinoma. In some embodiments, the neoplasm is a metastatic cancer. In some embodiments, the cancer is refractory to chemotherapy or radiotherapy, resistant to chemotherapy, or has relapsed.

In some embodiments, the subject has previously received one, two, three or more lines of therapy for the neoplasm. In some embodiments, the subject has not previously received treatment with a EGFR tyrosine kinase inhibitor. In some embodiments, the subject has received prior treatment with an EGFR tyrosine kinase inhibitor. In some embodiments, the administration and/or dosage of the drug combination is controlled to treat or prevent CNS metastases in the subject.

Another aspect of the patent document provides a method of treating or preventing CNS metastases in a subject, wherein the subject has been diagnosed to have a cancer comprising administering a drug combination described herein to the subject. In some embodiments, the cancer is non-small cell lung cancer (NSCLC). In some embodiments, the subject has been determined to have CNS metastases. In some embodiments, the subject has been determined to have no CNS metastases. In some embodiments, the subject has previously received one, two, three or more lines of therapy for the neoplasm. In some embodiments, the subject has not previously received treatment with an EGFR tyrosine kinase inhibitor.

### DETAILED DESCRIPTION

This patent document discloses a drug combination and methods of using the combination for treating cancers associated with overexpression or amplification of HER1, HER2, or HER4, or a mutant of HER1, HER2, or HER4. The administration of a therapeutically effective amount of the combination of the invention are advantageous over the individual component in that the combination provides one or more of the following improved properties when compared to the individual administration of a therapeutically effective amount of an individual component: i) a greater anticancer effect than the most active single agent, ii) synergistic or highly synergistic anticancer activity, iii) a dosing protocol that provides enhanced anticancer activity with a reduced side effect profile, iv) a reduction in the toxic effect profile, v) an increase in the therapeutic window, vi) an increase in the bioavailability of one or more of the components, or vii) an increase in apoptosis over the individual component.

While the following text may reference or exemplify specific embodiments of a drug combination or a method of using the combination treat a cancer, it is not intended to limit the scope of the combination or method to such particular reference or examples. Various modifications may be made by those skilled in the art, in view of practical and economic considerations, such as the amount of the individual component in the drug combination and the specific interval of administering the combination.

As used herein, the articles “a” and “an” refer to “one or more” or “at least one,” unless otherwise indicated. That is, reference to any element or component of an embodiment by the indefinite article “a” or “an” does not exclude the possibility that more than one element or component is present.

As used herein, the term “about” generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4. As used herein, the term “and/or” includes any and all combinations of one or more of the associated listed items. Expressions such as “at least one of,” when preceding a list of elements, modify the entire list of elements and do not modify the individual elements of the list. When referring to a dosing protocol, the term “day”, “per day” and the like, refer to a time within one calendar day which begins at midnight and ends at the following midnight.

By the term “treating” or “treatment” and any derivatives thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such a condition or biological manifestation thereof. Prophylactic therapy is appropriate, for example, when the subject is considered at high risk for developing cancer, such as when the subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Specific doses can be readily determined by one having ordinary skill in the art, using routine procedures

By the term “combination” as used herein is meant to include two or more drug components, which require either simultaneous administration or any manner of separate sequential administration of therapeutically effective amounts of the constituent drugs. Preferably, if the administration is not simultaneous, the constituent drugs are administered in a close time proximity to each other. Suitably, both drugs are administered within about 24, about 12, about 11, about 10, about 9, about 8, about 7, about 6, about 5, about 4, about 3, about 2, or about 1 hour(s) of each other. As used herein for example, when the administrations of poziotinib and Ramucirumab are less than about 45 minutes apart, this is considered to be simultaneous administration.

As used herein, the term “pharmaceutically acceptable carrier and/or excipient” refers to a carrier and/or excipient pharmacologically and/or physiologically compatible to a subject and an active component. A pharmaceutically acceptable carrier includes, without limitation, pH regulators, surfactants, adjuvants, and ionic strength enhancers. For example, pH regulators include, without limitation, phosphate buffer solutions; surfactants include, without limitation, cationic, anionic or nonionic surfactants, for example, Tween-80; ionic strength enhancers include, without limitation, sodium chloride.

As used herein, a “subject in need thereof” refers to a subject or patient suffering from a condition or disease that is associated with overexpression of HER1, HER2 or HER4 or any mutant thereof, who would benefit from the administration of a pharmaceutical combination comprising poziotinib and a VEGFR2 inhibitor (vascular endothelial growth factor receptor 2 inhibitor) such as Ramucirumab.

The term “wild-type” as used herein is understood in the art and refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild-type polypeptide or polynucleotide, respectively. Included in the term mutant is Single Nucleotide Polymorphism (SNP) where a single base pair distinction exists in the sequence of a nucleic acid strand compared to the most prevalently found (wild-type) nucleic acid strand. Cancers that are either wild-type or mutant for HER1, HER2, or HER4 or have amplification of HER1, HER2, or HER4 genes or have over expression of HER1, HER2, or HER4 protein are identified by known methods.

As used herein, the term “antibody” refers to an immune globulin usually consisting of two pairs of polypeptide chains (each pair has a light (L) chain and a heavy (H) chain). The antibody light chain can be classified as kappa light chain or lambda light chain. The heavy chain can be classified as mu, delta, gamma, alpha or epsilon, and isotypes of the antibody are separately defined as IgM, IgD, IgG, IgA and IgE. In light chain and heavy chain, variable region and constant region are linked via a “J” region with about 12 or more amino acids, and the heavy chain further contains a “D” region having about 3 or more amino acids. Each heavy chain consists of a heavy chain variable region (V H) and heavy constant region (CH). The heavy chain consists of 3 domains (CH1, CH2, and CH3). Each light chain consists of a light chain variable region (VL) and a light chain constant region (CL). The constant regions of the antibody can mediate immune globulin to bind to host tissues or factors, including various cells (e.g., effector cells) of the immune system and first component of the classical complement system.

As used herein, the term “antigen-binding fragment” of an antibody refers to a polypeptide containing a fragment of the full-length antibody, which fragment retains the ability to specifically bind to the same antigen to which the full-length antibody binds, and/or competes with the full-length antibody to specifically bind to the antigen.

As used herein, the term “subject” refers to as a human or an animal.

An aspect of the disclosure provides a drug combination including poziotinib or a pharmaceutically acceptable salt thereof, a vascular endothelial growth factor receptor (VEGFR) inhibitor, and one or more pharmaceutically acceptable carriers. Poziotinib, i.e., 1-[4-[4-(3,4-dichloro-2-fluoroanilino)-7-methoxyquinazolin-6-yl]oxypiperidin-1-yl]prop-2-en-1-one, has a structure represented by the formula 1.

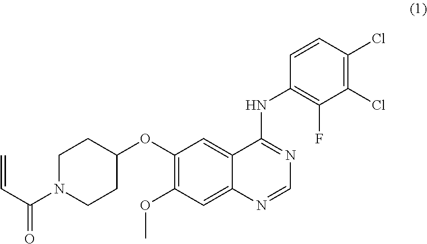 [SMILES: C=CC(=O)N1CCC(Oc2cc3c(Nc4ccc(Cl)c(Cl)c4F)ncnc3cc2OC)CC1]

The pharmaceutically acceptable salt form of poziobinib may include, without limitation, an acid-addition salt of an inorganic or organic acid. Examples of the inorganic acid addition salt may include salts of poziotinib formed from hydrochloric acid, hydrobromic acid, sulfuric acid, disulfuric acid, nitric acid, phosphoric acid, perchloric acid, or bromic acid; examples of the organic acid addition salt may include salts of formic acid, acetic acid, propionic acid, oxalic acid, succinic acid, benzoic acid, citric acid, maleic acid, malonic acid, malic acid, tartaric acid, gluconic acid, lactic acid, mandelic acid, glycolic acid, pyruvic acid, glutaric acid, ascorbic acid, palmitic acid, hydroxymaleic acid, hydroxybenzoic acid, phenylacetic acid, cinnamic acid, methanesulfonic acid, benzenesulfonic acid, toluenesulfonic acid, ethanedisulfonic acid, gestisic acid, fumaric acid, lactobionic acid, salicylic acid, phthalic acid, embonic acid, aspartic acid, glutamic acid, camsylic acid, besylic acid, or acetylsalicylic acid (aspirin). In some embodiments, poziotinib is in a salt form of hydrochloric acid.

Vascular endothelial growth factor receptor inhibitor or “VEGFR inhibitor” refers to any agent that inhibits the activity of VEGF-specific tyrosine kinase receptors VEGFR1, VEGFR2, VEGFR3, or any combination thereof. Non-limiting examples of VEGFR inhibitors include axitinib, sunitinib, vatalanib, sorafenib, GW-786034, CP-547632, AG-013736, lenvatinib, motesanib, pazopanib, regorafenib, ramucirumab, CDP-791, or any combination thereof. In further embodiments, a MNK-specific inhibitor is used in combination with a VEGFR inhibitor and a PD-1 specific antibody or binding fragment thereof. In still further embodiments, a MNK-specific inhibitor is used in combination with a VEGFR inhibitor and a PD-L1 specific antibody or binding fragment thereof. In yet further embodiments, a MNK-specific inhibitor is used in combination with a VEGFR inhibitor and a CTLA4 specific antibody or binding fragment thereof, or fusion protein. In yet further embodiments, a MNK-specific inhibitor is used in combination with a VEGFR inhibitor and a LAG3 specific antibody or binding fragment thereof, or fusion protein.

In some embodiments, the VEGFR inhibitor (e.g sorafenib) is a VEGFR1 inhibitor, a VEGFR2 inhibitor, and/or a VEGFR3 inhibitor. In some embodiments, the VEGFR inhibitor is a VEGFR2 inhibitor. VEGFR2 inhibitors may be any protein or small molecule that specifically binds VEGFR2 and inhibits or reduces one or more VEGFR2 biological functions. By “specifically binds” is meant a molecule that recognizes and interacts with VEGFR2 but that does not substantially recognize and interact with other molecules. In some embodiments, the VEGFR2 inhibitors bind VEGFR2 with a KID less than 500, 100, 1.0, 0.1, 0.01, or 0.001 nM.

Examples of VEGFR2 inhibitors include antibodies, such as heavy chain antibodies, antibodies naturally devoid of light chains, single domain antibodies derived from conventional four-chain antibodies, engineered antibodies and single domain scaffolds other than those derived from antibodies. Non-limiting examples of VEGFR2 inhibitors include CDP-791 (UCB), Ramucirumab (IMC-1121b, ImClone Systems), and AVE-005 (VEGF trap, Regeneron Pharmaceuticals). Other examples of VEGFR2 inhibitors include moieties such as affibodies, afflins, anticalins, avimers, DARPins, microbodies, trans-bodies; or inhibitors that are derived from lipocalins, ankyrins, tetranectins, C-type lectin, Protein A, gamma-crystalline, cysteine knots, and transferrin. In some embodiments, the VEGFR2 inhibitor is Ramucirumab.

Another aspect of the disclosure provides a method of treating a neoplasm in a subject by administering the drug combination described herein. In some embodiments, poziotinib may be administered in an amount of 0.1 mg to 50 mg. For instance, poziotinib in a hydrochloride salt form, may be administered orally, such as in a tablet. The poziotinib may be administered in a dose of 4-25 mg, such as at a dose of 5, 6, 7, 8, 9, 10, 11, 12, 13, 14, 15, 16, 17, 18, 19, 20, 21, 22, 23, or 24 mg. The dosing may be daily, every other day, every 3 days or weekly. The dosing may be on a continuous schedule, such as on 28 days cycles.

In some embodiments, the VEGFR2 inhibitor may be administered in an amount of 0.5 to 10 mg per kg of patient's body weight. In some embodiments, the VEGFR2 inhibitor is administered in an amount of 1.5 to 5.5 mg per kg of body weight. In some embodiments, the VEGFR2 inhibitor is Ramucirumab.

The drug combination described herein can be administered either, simultaneously, or by any manner of separate sequential administration of therapeutically effective amounts of the constituent drugs—poziotinib in combination with a VEGFR2 inhibitor (e.g. Ramucirumab) or the pharmaceutically acceptable salts or solvates thereof. Preferably, if the administration is not simultaneous, the compounds are administered in a close time proximity to each other. Furthermore, it does not matter if the constituent drugs are administered in the same dosage form, e.g. one compound may be administered topically and the other compound may be administered orally. In some embodiments, both compounds are administered orally.

In some embodiments, the drug combination is administered within a “specified period”. By the term “specified period” and derivatives thereof, as used herein is meant the interval of time between the administration of one of the constituent drug of the inventive combination and another constituent drug. Unless otherwise defined, the specified period can include simultaneous administration. In an embodiment of a two constituent drug combination, when both compounds of the invention are administered once a day the specified period refers to timing of the administration of poziotinib and the other, in the relevant order during a single day. When one or both constituent drugs are administered more than once a day, the specified period is calculated based on the first administration of each compound on a specific day. All administrations of a compound of the invention that are subsequent to the first during a specific day are not considered when calculating the specific period.

In some embodiments, if the constituent drugs of the combination are administered within a “specified period” and not administered simultaneously, they are both administered within about 24, 12, 11, 10, 9, 8, 7, 6, 5, 4, 3, 2, or 1 hour(s) of each other—in this case, the specified period will be about 24 12, 11, 10, 9, 8, 7, 6, 5, 4, 3, 2, or 1 hour(s). As used herein, for an embodiment of a two constituent drug combination, the administration of poziotinib and the other constituent drug in less than about 45 minutes apart is considered simultaneous administration.

In some embodiments, when the combination is administered for a “specified period”, the compounds will be co-administered for a “duration of time”. By the term “duration of time” and derivatives thereof, as used herein is meant that both constituent drugs are administered within a “specified period” for an indicated number of consecutive days, optionally followed by a number of consecutive days where only one of the component compounds is administered.

Regarding “specified period” administration, in at least one embodiment, during the course of treatment, both constituent drugs will be administered within a specified period for at least 1, 2, 3, 5, 7, 14, or 30 day(s)—in this case, the duration of time will be at least 1, 2, 3, 5, 7, 14, or 30 day(s). When, during the course of treatment, both constituent drugs are administered within a specified period for over 30 days, the treatment is considered chronic treatment and will continue until an altering event, such as a reassessment in cancer status or a change in the condition of the patient, warrants a modification to the protocol.

Further regarding “specified period” administration, in another embodiment, during the course of treatment with a two constituent drug combination both constituent drugs will be administered within a specified period for at least 1 day, followed by the administration of poziotinib alone for at least 1, 2, 3, 4, 5, 6, or 7 day(s)—in this case, the duration of time will be at least 2, 3, 4, 5, 6, 7, or 8 days; suitably, during the course of treatment, both compounds will be administered within a specified period for at least 2 consecutive days, followed by administration of poziotinib alone for at least 1 day, 2, 3, 4, 5, 6, or 7 consecutive days—in this case, the duration of time will be at least 3, 4, 5, 6, 7, 8, or 9 days; suitably, during the course of treatment, both constituent drugs will be administered within a specified period for at least 3 consecutive days, followed by administration of poziotinib alone for at least 1 day, 2, 3, 4, 5, 6, or 7 consecutive days—in this case, the duration of time will be at least 4, 5, 6, 7, 8, 9, or 10 days; suitably, during the course of treatment, both constituent drugs will be administered within a specified period for at least 4 consecutive days, followed by administration of poziotinib alone for at least 1 day, 2, 3, 4, or 7 consecutive days—in this case, the duration of time will be at least 5, 6, 7, 8 or 11 days; suitably, during the course of treatment, both constituent drugs will be administered within a specified period for at least 5 consecutive days, followed by administration of poziotinib alone for at least 1 day, 2, 3, 4, or 5 consecutive days—in this case, the duration of time will be at least 6, 7, 8, 9, or 10 days. In another embodiment, during the course of treatment, both constituent drugs will be administered within a specified period for from 1 to 3 consecutive days, followed by administration of poziotinib alone for from 3 to 7 consecutive days.

In some embodiments, during the course of treatment, both constituent drugs will be administered within a specified period for from 3 to 6 consecutive days, followed by administration of poziotinib alone for from 1 to 4 consecutive days. In some embodiments, during the course of treatment, both constituent drugs will be administered within a specified period for 2 consecutive days, followed by administration of poziotinib alone for from 3 to 7 consecutive days. In some embodiments, during the course of treatment, both constituent drugs will be administered within a specified period for from 1 to 3 days over a 7 day period, and during the other days of the 7 day period poziotinib will be administered alone. In yet another embodiment, during the course of treatment, both constituent drugs will be administered within a specified period for 2 days over a 7 day period, and during the other days of the 7 day period poziotinib will be administered alone.

Further regarding “specified period” administration, during the course of treatment, for an embodiment of a two constituent drug combination both will be administered within a specified period for at least 1 day, followed by the administration of the other constituent drug alone for at least 1, 2, 3, 4, 5, 6, or 7 day(s)—in this case, the duration of time will be at least 2, 3, 4, 5, 6, 7, or 8 days; suitably, during the course of treatment, both constituent drugs will be administered within a specified period for at least 2 consecutive days, followed by administration of the other constituent drug alone for at least 1 day, 2, 3, 4, 5, 6, or 7 consecutive days—in this case, the duration of time will be at least 3, 4, 5, 6, 7, 8, or 9 days; suitably, during the course of treatment, both constituent drugs will be administered within a specified period for at least 3 consecutive days, followed by administration of the other constituent drug alone for at least 1 day, 2, 3, 4, 5, 6, or 7 consecutive days—in this case, the duration of time will be at least 4, 5, 6, 7, 8, 9, or 10 days; suitably, during the course of treatment, both constituent drugs will be administered within a specified period for at least 4 consecutive days, followed by administration of the other constituent drug alone for at least 1 day, 2, 3, 4, or 7 consecutive days—in this case, the duration of time will be at least 5, 6, 7, 8 or 11 days; suitably, during the course of treatment, both constituent drugs will be administered within a specified period for at least 5 consecutive days, followed by administration of the other constituent drug alone for at least 1 day, 2, 3, 4, or 5 consecutive days in this case, the duration of time will be at least 6, 7, 8, 9, or 10 days. Suitably, during the course of treatment, both constituent drugs will be administered within a specified period for from 1 to 3 consecutive days, followed by administration of the other constituent drug alone for from 3 to 7 consecutive days. Suitably, during the course of treatment, both constituent drugs will be administered within a specified period for from 3 to 6 consecutive days, followed by administration of the other constituent drug alone for from 1 to 4 consecutive days. Suitably, during the course of treatment, both constituent drugs will be administered within a specified period for 2 consecutive days, followed by administration of the other constituent drug alone for from 3 to 7 consecutive days. Suitably, during the course of treatment, both constituent drugs will be administered within a specified period for from 1 to 3 days over a 7 day period, and during the other days of the 7 day period the other constituent drug will be administered alone. Suitably, during the course of treatment, both constituent drugs will be administered within a specified period for 2 days over a 7 day period, and during the other days of the 7 day period the other constituent drug will be administered alone.

Further regarding “specified period” administration, during the course of treatment, for an embodiment of a two constituent drug combination, poziotinib and the other constituent drug (e.g. Ramucirumab) will be administered within a specified period for from 1 to 3 days over a 7 day period, and during the other days of the 7 day period poziotinib will be administered alone. Suitably, this 7 day protocol is repeated for 2 cycles or for 14 days; suitably for 4 cycles or 28 days; suitably for continuous administration.

In some embodiments, during the course of treatment, for an embodiment of a two constituent drug combination, poziotinib and the other constituent drug will be administered within a specified period for from 1 to 3 days over a 7 day period, and during the other days of the 7 day period the other constituent drug will be administered alone. In some embodiments, this 7 day protocol is repeated for 2 cycles or for 14 days; suitably for 4 cycles or 28 days; suitably for continuous administration.

In some embodiments, during the course of treatment, for an embodiment of a two constituent drug combination, poziotinib and the other constituent drug will be administered within a specified period for from 1 to 5 days over a 14 day period, and during the other days of the 14 day period poziotinib will be administered alone. In some embodiments, this 14 day protocol is repeated for 2 cycles or for 28 days; suitably for continuous administration.

In some embodiments, during the course of treatment, for an embodiment of a two constituent drug combination, poziotinib and the other constituent drug will be administered within a specified period for from 1 to 5 days over a 14 day period, and during the other days of the 14 day period the other constituent drug will be administered alone. In some embodiments, this 14 day protocol is repeated for 2 cycles or for 28 days; suitably for continuous administration.

In some embodiments, if the constituent drugs are not administered during a “specified period”, they are administered sequentially. By the term “sequential administration” as used herein is meant for example for an embodiment of a two constituent drug combination, that one of poziotinib and the other constituent drug is administered for 1 or more consecutive days and the other of poziotinib and the other constituent drug is subsequently administered for 1 or more consecutive days. Unless otherwise defined, the “sequential administration” and in all dosing protocols described herein in the case of a two constituent drug combination, do not have to commence with the start of treatment and terminate with the end of treatment, it is only required that the administration of one of poziotinib and the other constituent drug followed by the administration of the other, or the indicated dosing protocol, occur at some point during the course of treatment. Also, contemplated herein is a drug holiday utilized between the sequential administration of one of poziotinib and the other constituent drug and the other. As used herein, a drug holiday is a period of days after the sequential administration of one of poziotinib and the other constituent drug and before the administration of the other where neither poziotinib nor the other constituent drug is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Regarding Sequential Administration, in some embodiments for a two constituent drug combination, one of poziotinib and the other constituent drug is administered for from 1 to 30 consecutive days, followed by an optional drug holiday, followed by administration of the other for from 1 to 30 consecutive days.

In some embodiments of a two constituent drug combination, the other constituent drug will be administered first in the sequence, followed by an optional drug holiday, followed by administration of poziotinib.

In some embodiments, poziotinib will be administered first in the sequence, followed by an optional drug holiday, followed by administration of the other constituent drug.

It is understood that a “specified period” administration and a “sequential” administration can be followed by one or more cycles of repeat dosing or can be followed by an alternate dosing protocol, and a drug holiday may precede the repeat dosing or alternate dosing protocol.

In some embodiments, the amount of poziotinib administered as part of the combination according to the present invention will be an amount selected from about 0.1 mg to about 50 mg; suitably, the amount will be selected from about 0.5 mg to about 50 mg; suitably, the amount will be selected from about 1 mg to about 50 mg; suitably, the amount will be selected from about 5 mg to about 50 mg; suitably, the amount will be selected from about 1 mg to about 30 mg; suitably, the amount will be selected from about 5 mg to about 20 mg; suitably, the amount will be selected from about 1 mg to about 10 mg; suitably, the amount will be selected from about 0.1 mg to about 5 mg; suitably, the amount will be 1 mg, suitably, the amount will be 5 mg, suitably, the amount will be 10 mg, suitably, the amount will be 20 mg, suitably, the amount will be 30 mg; suitably, the amount will be 50 mg. Accordingly, the amount of poziotinib administered as part of the combination according to the present invention will be an amount selected from about 0.1 mg to about 50 mg. For example, the amount of poziotinib administered as part of the combination according to the present invention is suitably selected from 1 mg, 5 mg, 10 mg, 20 mg, 30 mg and 50 mg. In some embodiments, the selected amount of poziotinib is administered from 1 to 4 times a day, in one or more tablets. In some embodiments, the selected amount of poziotinib is administered twice a day, in one or more tablets. In some embodiments, the selected amount of poziotinib is administered once a day, in one or more tablets. In some embodiments, the administration of poziotinib will begin as a loading dose. In some embodiments, the loading dose will be an amount from 2 to 100 times the maintenance dose; suitably from 2 to 10 times; suitably from 2 to 5 times; suitably 2 times; suitably 3 times; suitably 4 times; suitably 5 times. In some embodiments, the loading does will be administered from 1 to 7 days; suitably from 1 to 5 days; suitably from 1 to 3 days; suitably for 1 day; suitably for 2 days; suitably for 3 days, followed by a maintenance dosing protocol.

In some embodiments, the amount of the second constituent drug that is not poziotinib administered as part of the combination according to the present invention will be an amount selected from about 0.1 mg to about 3,500 mg/m², from about 0.5 mg to about 3,500 mg/m²; suitably, the amount will be selected from about 1.0 mg to about 3,500 mg/m²; suitably, the amount will be selected from about 10.0 mg to about 3,500 mg/m²; suitably, the amount will be selected from about 50.0 mg to about 3,500 mg/m²; suitably, the amount will be selected from about 100.0 mg to about 3,500 mg/m²; suitably, the amount will be selected from about 100.0 mg to about 3,000 mg/m²; suitably, the amount will be selected from about 0.1 mg to about 20 mg/m²; suitably, the amount will be selected from about 0.5 mg to about 10 mg/m²; suitably, the amount will be selected from about 0.5 mg to about 10 mg/kg; suitably, the amount will be selected from about 0.5 mg to about 50 mg/m²; suitably, the amount will be selected from about 50 mg to about 1,000 mg/m²; suitably, the amount will be selected from about 100 mg to about 500 mg/m²; suitably, the amount will be selected from about 100 mg to about 300 mg/m²; suitably, the selected amount of the second constituent drug that is not poziotinib is administered from 1 to 4 times a day. In some embodiments, the selected amount of the second constituent drug is administered 1 to 4 times a day.

As used herein, all amounts specified for poziotinib and the other constituent drug(s) are indicated as the administered amount of free or unsalted and unsolvated compound per dose.

The constituent therapeutic agents/drugs of the combination of the methods described herein can be administered to a subject in need sequentially or simultaneously. The constituent or the combination can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition of a constituent or a combination described herein can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

In any embodiment disclosed herein, the VEGFR2 inhibitor can be Ramucirumab.

Another aspect of the document provides a kit for treating a neoplasm associated with overexpression or amplification of EGFR, HER1, HER2, or HER4 or a mutant of EGFR, HER1, HER2, or HER4 in a subject, the kit including a first part and a second part, wherein the first part includes poziotinib, and the second part includes at least a VEGFR2 inhibitor (e.g. Ramucirumab). The kit can also further comprise a package insert comprising instructions for treating a neoplasm associated with overexpression or amplification of EGFR, HER1, HER2, or HER4 or a mutant thereof in a subject.

The constituent drugs of the kit may be provided in a form which is suitable for sequential, separate and/or simultaneous administration. The kit can also be provided with instruction, such as dosage and administration instructions. Such dosage and administration instructions can be of the kind that is provided to a doctor, for example by a drug product label, or they can be of the kind that is provided by a doctor, such as instructions to a patient.

Various combinations may be employed. For the example below poziotinib or VEGFR2 inhibitor (e.g. Ramucirumab) is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

In some embodiments, the administration regimen includes a break, or rest period, between the sequential administration of one of poziotinib and the other constituent drug, and the other drug. As used herein, a drug break (rest period) is a period of days after the sequential administration of one of poziotinib and the other constituent drug, and before the administration of the other where neither poziotinib nor the other constituent drug is administered. Suitably the drug holiday is a period of days selected from: 1 day, 2 days, 3 days, 4 days, 5 days, 6 days, 7 days, 8 days, 9 days, 10 days, 11 days, 12 days, 13 days and 14 days.

The method disclosed herein is applicable to the treatment of a cancer which is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In some embodiments, the cancer is non-small-cell lung carcinoma (NSCLC).

In some embodiments, the method includes screening the subject in need of treatment and determining wild-type or mutant EGFR, HER1, HER2, and HER4 tumor cells. The specific mutations can be identified by DNA amplification and sequencing techniques, DNA and RNA detection techniques, including, without limitation, Northern and Southern blot, respectively, and/or various biochip and array technologies or in-situ hybridization. Wild type and mutant polypeptides can be detected by a variety of techniques including, without limitation, immunodiagnostic techniques such as ELISA, Western blot or immunocytochemistry.

The sample to be analyzed can be any bodily tissue or fluid that includes nucleic acids from the lung cancer in the subject. In certain embodiments, the sample will be a blood sample comprising circulating tumor cells or cell free DNA. In other embodiments, the sample can be a tissue, such as a lung tissue. The lung tissue can be from a tumor tissue and may be fresh frozen or formalin-fixed, paraffin-embedded (FFPE). In certain embodiments, a lung tumor FFPE sample is obtained.

Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine, tumor, or expectorant. The sample itself will typically include nucleated cells (e.g., blood or buccal cells) or tissue removed from the subject including normal or tumor tissue. Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of EGFR, HER1, HER2, or HER4 exon mutations, as described herein can be determined using methods known in the art. For example, gel electrophoresis, capillary electrophoresis, size exclusion chromatography, sequencing, and/or arrays can be used to detect the presence or absence of insertion mutations. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an insertion mutation as described herein. An insertion mutation can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular variant.

A set of probes typically refers to a set of primers, usually primer pairs, and/or detectably-labeled probes that are used to detect the target genetic variations (e.g., EGFR or HER2 exon 20 mutations) used in the actionable treatment recommendations of the present disclosure. The primer pairs are used in an amplification reaction to define an amplicon that spans a region for a target genetic variation for each of the aforementioned genes. The set of amplicons are detected by a set of matched probes. In an exemplary embodiment, the present methods may use TaqMan™ (Roche Molecular Systems, Pleasanton, Calif.) assays that are used to detect a set of target genetic variations, such as EGFR or HER2 exon 20 mutations. In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed.

Analysis of nucleic acid markers can be performed using techniques known in the art including, without limitation, sequence analysis, and electrophoretic analysis. Non-limiting examples of sequence analysis include Maxam-Gilbert sequencing, Sanger sequencing, capillary array DNA sequencing, thermal cycle sequencing (Sears et al., 1992), solid-phase sequencing (Zimmerman et al., 1992), sequencing with mass spectrometry such as matrix-assisted laser desorption/ionization time-of-flight mass spectrometry (MALDI-TOF/MS; Fu et al., 1998), and sequencing by hybridization (Chee et al., 1996; Drmanac et al., 1993; Drmanac et al., 1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

Other methods of nucleic acid analysis can include direct manual sequencing (Church and Gilbert, 1988; Sanger et al., 1977; U.S. Pat. No. 5,288,644); automated fluorescent sequencing; single-stranded conformation polymorphism assays (SSCP) (Schafer et al., 1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); denaturing high performance liquid chromatography (DHPLC, Underhill et al., 1997); infrared matrix-assisted laser desorption/ionization (IR-MALDI) mass spectrometry (WO 99/57318); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

In one example, a method of identifying a EGFR, HER1, HER2, or HER4 mutation in a sample comprises contacting a nucleic acid from said sample with a nucleic acid probe that is capable of specifically hybridizing to nucleic acid encoding a mutated HER2 protein, or fragment thereof incorporating a mutation, and detecting said hybridization. In a particular embodiment, said probe is detectably labeled such as with a radioisotope (³H, ³²P, or ³³P), a fluorescent agent (rhodamine, or fluorescein) or a chromogenic agent. In a particular embodiment, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, the probes of the present disclosure are provided in a kit for identifying EGFR or HER2 mutations in a sample, where the kit comprises an oligonucleotide that specifically hybridizes to or adjacent to a site of mutation in the EGFR or HER2 gene. The kit may further comprise instructions for treating patients having tumors that contain EGFR or HER2 insertion mutations with poziotinib or afatinib based on the result of a hybridization test using the kit.

In some embodiments, the method includes determining the neoplasm in the subject to be associated with overexpression or amplification of at least one gene of EGFR, HER1, HER2, and HER4 or a mutant thereof and may be an abnormal growth of tissue, which if it forms a mass, is commonly referred to as a tumor having overexpression of at least one of EGFR, HER1, HER2, HER4 and mutant thereof or amplification of at least one gene coding of EGFR, HER1, HER2, HER4 or mutant thereof. In some embodiments, the neoplasm to be treated with the method disclosed herein is associated mutants which can be present in any of exon 18, 19, 20 and 21 or any combinations thereof. For example, the mutant may be HER1 having exon 19 deletion, T790M substitution, L828R substitution, or combination thereof. In another embodiment, the mutation may be at HER2 exon 20, such as exon 20 insertion mutations. In another embodiment, there may be one or more mutations in exon 19 or 20 or both. For example, the one or more EGFR exon 20 mutations comprise one or more point mutations, insertions, and/or deletions of 3-18 nucleotides between amino acids 763-778.

In some embodiments, the method includes determining the neoplasm in the subject to be associated with an EGFR or HER2 mutation within one or more of exon 18, exon 19, exon 20 and exon 21. In some embodiments, the cancer is associated with EGFR or HER2 mutation occurring in exon 20 mutation. The EGFR or HER2 exon 20 mutation may suitably comprise a EGFR or HER2 in-frame exon 20 insertion mutation, a EGFR or HER2 exon 20 point mutation, or any combination thereof. The HER2 in-frame exon 20 insertion mutation may be selected from the group consisting of A775_G776insYVMA, G776_V777insVC, P780_Y781insGSP, and combinations thereof. The HER2 exon 20 point mutation may be selected from the group consisting of L775S, G776V, V777L, and combinations thereof. In some embodiments, the HER2 exon 20 mutation is not a T790M point mutation.

In some embodiments, the method includes determining the neoplasm in the subject to be associated with 2, 3, or 4 EGFR exon 20 mutations at one or more residues selected from the group consisting of A763, A767, 5768, V769, D770, N771, P772, and H773. In some embodiments, the subject may be determined to not have an EGFR mutation at residue C797. In some embodiments, the one or more EGFR mutations include substitution and/or deletion at the A763, A767, S768, V769, D770, N771, P772, and H773 in exon 20. In some embodiments, the one or more exon 20 mutations are selected from the group consisting of A763insFQEA, A767insASV, S768dupSVD, V769insASV, D770insSVD, D770insNPG, H773insNPH, N771del insGY, N771del insFH, and N771dupNPH.

In some embodiments, the method includes determining the neoplasm in the subject to be associated with HER2 mutation occurring in exon 21 mutation. The HER2 exon 21 mutation(s) may comprise one or more point mutations, insertions, and/or deletions of 1-18 nucleotides between amino acids 832-883. In some embodiments, the one or more HER2 exon 21 mutations are at one or more residues selected from the group consisting of V842, R868, and L869. In some embodiments, the one or more exon 21 mutations are selected from the group consisting of V8421, R868W, and L869R. In some embodiments, the one or more HER2 exon 21 mutations are at one or more residues selected from the group consisting of V842 and R868. In some embodiments, the one or more exon 21 mutations are selected from the group consisting of V8421 and R868W.

In some embodiments, the method includes determining the neoplasm in the subject to be associated with one or more HER2 exon 20 mutations. In some embodiments, HER2 exon 20 mutation may comprise one or more point mutations, insertions, and/or deletions of 1-18, such as 3-18, nucleotides between amino acids 770-786. In some embodiments, the one or more HER2 exon 20 mutations may be at residue Y772, V773, A775, G776, V777, G778, S779, and/or P780. In some embodiments, the method includes determining the subject to be associated with one or more HER2 exon 20 mutations selected from the group consisting of A775insV G776C, A775insYVMA, G776C V777insC, G776del insVV, G776del insVC, P780insGSP, Y772dupYVMA, V773M, G776delinsLC, V777L, V777insCG, G778dupGSP, P780insGSP, L786V, G776delinsVC and G778insLPS.

In some embodiments, the method includes determining the neoplasm in the subject to be associated with one or more EGFR exon 20 mutations selected from the group consisting of A763insFQEA, A767insASV, S768dupSVD, V769insASV, D770insSVD, D770insNPG, H773insNPH, N771del insGY, N771del insFH, N771dupNPH, A767insTLA, 57681, V769L, V769insGSV, D770del insGY, D770insG, D770insY H773Y, N771insHH, P772insDNP, H773insAH, H773insH, V774insHV, S784F, R776C, V774M, V769M, G796D, S784F, C775Y, S811F, T790M, V774A, D770A.

In some embodiments, the subject may have or develop a mutation at EGFR residue C797 which may result in resistance to the TKI, such as poziotinib. Thus, in some embodiments, the method includes determining that the subject does not have a mutation at EGFR C797 and/or T790, such as C797S and/or T790M.

A further aspect of the patent document is directed to a method of using the drug combination described herein for improving the adverse events profile in a subject undergoing treatment for a cancer associated with overexpression or amplification of HER2, or a mutant of HER2, the method comprising the steps of a) in a 21-day cycle±3 days, i) administering a single dose of Ramucirumab; and ii) administering a daily dose of poziotinib; and b) optionally repeating the cycle, where the adverse effect is selected from the group consisting of cardiotoxicity hematologic toxicities, diarrhea, skin rash, mucositis, fatigue, electrolyte abnormalities and hepatotoxicity. In some embodiments, the cancer is NSCLC.

Another aspect of the patent document is directed to a method of using the drug combination described herein for treating cancer in a subject, where the cancer is associated with overexpression or amplification of EGFR, HER1, HER2, or HER4, or a mutant of HER1, HER2, or HER4. In some embodiments, the combination comprises therapeutically effective amounts of poziotinib and Ramucirumab, where poziotinib is administered orally and Ramucirumab is administered by IV infusion. In some embodiments, the cancer is NSCLC.

Another aspect of the patent document is directed to a method of treating or preventing CNS metastases in a subject, wherein the subject has been diagnosed to have a cancer. The method includes administering the drug combination described herein. In some embodiments, the CNS metastases is brain metastases. In some embodiments, the cancer is non-small cell lung cancer (NSCLC). In some embodiments, the subject has been determined to have CNS metastases. In some embodiments, the subject has been determined to have no CNS metastases. In some embodiments, the combination comprises therapeutically effective amounts of poziotinib and Ramucirumab, where poziotinib is administered orally and Ramucirumab is administered by IV infusion. In some embodiments, the cancer is NSCLC.

In some embodiments of any of the methods described herein, the cancer is determined to be locally advanced or metastatic. Examples of cancers that can be defined as metastatic include but are not limited to non-small cell lung cancer, breast cancer, ovarian cancer, colorectal cancer, biliary tract cancer, bladder cancer, brain cancer including glioblastomas and medullablastomas, cervical cancer, choriocarcinoma, endometrial cancer, esophageal cancer, gastric cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

In some embodiments, the subject has received a prior treatment including for example, chemotherapy, radiotherapy, surgery, and other antiproliferative agent or immunotherapy. In some embodiments, the subject has not received a prior treatment such as chemotherapy, radiotherapy, surgery, and other antiproliferative agent or immunotherapy.

In some embodiments of any of the above described methods, the cancer is refractory to chemotherapy or radiotherapy. In some embodiments, the cancer is resistant to chemotherapy. In some embodiments, the cancer has relapsed. In some embodiments, the cancer is metastatic.

In some embodiments of any of the methods described herein, the cancer is resistant to an antiproliferative agent. Non-limiting examples of the antiproliferative agent include alkylating agents, platinum agents, antimetabolites, topoisomerase inhibitors, antitumor antibiotics, antimitotic agents, aromatase inhibitors, thymidylate synthase inhibitors, dna antagonists, farnesyltransferase inhibitors, pump inhibitors, histone acetyltransferase inhibitors, metalloproteinase inhibitors, ribonucleoside reductase inhibitors, tnf alpha agonists/antagonists, endothelin a receptor antagonist, retinoic acid receptor agonists, immuno-modulators, hormonal and antihormonal agents, photodynamic agents, and kinase inhibitors.

In some embodiments of any of the above described methods, there may be included an additional treatment, including for example, chemotherapy, radiotherapy, surgery, and other suitable immunotherapy. It is contemplated that other antiproliferative agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

Non-limiting examples of the antiproliferative agent in any of the above embodiments include alkylating agents: Busulfan, dacarbazine, ifosfamide, hexamethylmelamine, thiotepa, dacarbazine, lomustine, chlorambucil, procarbazine, altretamine, estramustine phosphate, mechlorethamine, streptozocin, temozolomide, Semustine cyclophosphamide; platinum agents: spiroplatin, tetraplatin, ormaplatin, iproplatin, ZD-0473 (AnorMED), oxaliplatin carboplatin, lobaplatin (Aeterna), satraplatin (Johnson Matthey), BBR-3464 (Hoffmann-La Roche), SM-11355 (Sumitomo), AP-5280 (Access), cisplatin, arboplatin, cisplatin, satraplatin, oxaliplatin, nedaplatin, triplatin tetranitrate, temozolomide, procarbazin; antimetabolites: azacytidine, Floxuridine, 2-chlorodeoxyadenosine, 6-mercaptopurine, 6-thioguanine, cytarabine, 2-fluorodeoxy cytidine, methotrexate, tomudex, fludarabine, raltitrexed, trimetrexate, deoxycoformycin, pentostatin, hydroxyurea, decitabine (SuperGen), clofarabine (Bioenvision), irofulven (MGI Pharma), DMDC (Hoffmann-La Roche), ethynylcytidine (Taiho) gemcitabine, capecitabine;

topoisomerase inhibitors: amsacrine, epirubicin, etoposide, teniposide or mitoxantrone, 7-ethyl-10-hydroxy-camptothecin, dexrazoxanet (TopoTarget), pixantrone (Novuspharma), rebeccamycin analogue (Exelixis), BBR-3576 (Novuspharma), rubitecan (SuperGen), irinotecan (CPT-11), topotecan;
antitumor antibiotics: valrubicin, therarubicin, idarubicin, rubidazone, plicamycin, porfiromycin mitoxantrone (novantrone), amonafide, azonafide, anthrapyrazole, oxantrazole, losoxantrone, MEN-10755 (Menarini), GPX-100 (Gem Pharmaceuticals), Epirubicin, mitoxantrone, doxorubicin;
antimitotic agents: colchicine, vinblastine, vindesine, dolastatin 10 (NCI), rhizoxin (Fujisawa), mivobulin (Warner-Lambert), cemadotin (BASF), RPR 109881A (Aventis), TXD 258 (Aventis), epothilone B (Novartis), T 900607 (Tularik), T 138067 (Tularik), cryptophycin 52 (Eli Lilly), vinflunine (Fabre), auristatin PE (Teikoku Hormone), BMS 247550 (BMS), BMS 184476 (BMS), BMS 188797 (BMS), taxoprexin (Protarga), SB 408075 (GlaxoSmithKline), Vinorelbine, Trichostatin A, E7010 (Abbott), PG-TXL (Cell Therapeutics), IDN 5109 (Bayer), A 105972 (Abbott), A 204197 (Abbott), LU 223651 (BASF), D 24851 (ASTAMedica), ER-86526 (Eisai), combretastatin A4 (BMS), isohomohalichondrin-B (PharmaMar), ZD 6126 (AstraZeneca), AZ10992 (Asahi), IDN-5109 (Indena), AVLB (Prescient NeuroPharma), azaepothilone B (BMS), BNP-7787 (BioNumerik), CA-4 prodrug (OXiGENE), dolastatin-10 (NIH), CA-4 (OXiGENE), docetaxel, vincristine, paclitaxel;
aromatase inhibitors: aminoglutethimide, atamestane (BioMedicines), letrozole, anastrazole, YM-511 (Yamanouchi), formestane, exemestane;
thymidylate synthase inhibitors: pemetrexed (Eli Lilly), ZD-9331 (BTG), nolatrexed (Eximias), CoFactor™ (BioKeys);
dna antagonists: trabectedin (PharmaMar); glufosfamide (Baxter International), albumin+32P (Isotope Solutions), thymectacin (NewBiotics), edotreotide (Novartis), mafosfamide (Baxter International), apaziquone (Spectrum Pharmaceuticals), 06 benzyl guanine (Paligent); farnesyltransferase inhibitors: arglabin (NuOncology Labs), lonafarnib (Schering-Plough), BAY-43-9006 (Bayer), tipifarnib (Johnson & Johnson), perillyl alcohol (DOR BioPharma); pump inhibitors: CBT-1 (CBA Pharma), tariquidar (Xenova), MS-209 (Schering AG), zosuquidar trihydrochloride (Eli Lilly), biricodar dicitrate (Vertex);
histone acetyltransferase inhibitors: tacedinaline (Pfizer), SAHA (Aton Pharma), MS-275 (Schering AG), pivaloyloxymethyl butyrate (Titan), depsipeptide (Fujisawa);
metalloproteinase inhibitors: Neovastat (Aeterna Laboratories), marimastat (British Biotech), CMT-3 (CollaGenex), BMS-275291 (Celltech);
ribonucleoside reductase inhibitors: gallium maltolate (Titan), triapine (Vion), tezacitabine (Aventis), didox (Molecules for Health);
tnf alpha agonists/antagonists: virulizin (Lorus Therapeutics), CDC-394 (Celgene), revimid (Celgene);
endothelin a receptor antagonist: atrasentan (Abbott), ZD-4054 (AstraZeneca), YM-598 (Yamanouchi);
retinoic acid receptor agonists: fenretinide (Johnson & Johnson), LGD-1550 (Ligand), alitretinoin (Ligand);
immuno-modulators: Pembrolizumab (formerly lambrolizumab, brand name Keytruda); interferon, oncophage (Antigenics), GMK (Progenics), adenocarcinoma, vaccine (Biomira), CTP-37 (AVI BioPharma), IRX-2 (Immuno-Rx), PEP-005 (Peplin Biotech), synchrovax vaccines (CTL Immuno), melanoma vaccine (CTL Immuno), p21 RAS vaccine (GemVax), MAGE-A3 (GSK), nivolumab (BMS), abatacept (BMS), dexosome therapy (Anosys), pentrix (Australian Cancer Technology), ISF-154 (Tragen), cancer vaccine (Intercell), norelin (Biostar), BLP-25 (Biomira), MGV (Progenics), β-alethine (Dovetail), CLL therapy (Vasogen), Ipilimumab (BMS), CM-10 (cCam Biotherapeutics), MPDL3280A (Genentech);
hormonal and antihormonal agents: estrogens, conjugated estrogens, ethinyl estradiol, chlortrianisen, idenestrol, hydroxyprogesterone caproate, medroxyprogesterone, testosterone, testosterone propionate, fluoxymesterone, methyltestosterone, diethylstilbestrol, megestrol, bicalutamide, flutamide, nilutamide, dexamethasone, prednisone, methylprednisolone, prednisolone, aminoglutethimide, leuprolide, octreotide, mitotane, P-04 (Novogen), 2-methoxyestradiol (EntreMed), arzoxifene (Eli Lilly), tamoxifen, toremofine, goserelin, Leuporelin, bicalutamide;
photodynamic agents: talaporfin (Light Sciences), Theralux (Theratechnologies), motexafin gadolinium (Pharmacyclics), Pd-bacteriopheophorbide (Yeda), lutetium texaphyrin (Pharmacyclics), hypericin; and
kinase inhibitors: afatinib, osimertinib, imatinib (Novartis), leflunomide (Sugen/Pharmacia), ZD1839 (AstraZeneca), erlotinib (Oncogene Science), canertinib (Pfizer), squalamine (Genaera), SU5416 (Pharmacia), SU6668 (Pharmacia), ZD4190 (AstraZeneca), ZD6474 (AstraZeneca), vatalanib (Novartis), PKI166 (Novartis), GW2016 (GlaxoSmithKline), EKB-509 (Wyeth), trastuzumab (Genentech), OSI-774 (Tarceva™), CI-1033 (Pfizer), SU11248 (Pharmacia), RH3 (York Medical), Genistein, Radicinol, Met-MAb (Roche), EKB-569 (Wyeth), kahalide F (PharmaMar), CEP-701 (Cephalon), CEP-751 (Cephalon), MLN518 (Millenium), PKC412 (Novartis), Phenoxodiol (Novogen), C225 (ImClone), rhu-Mab (Genentech), MDX-H210 (Medarex), 2C4 (Genentech), MDX-447 (Medarex), ABX-EGF (Abgenix), IMC-1C11 (ImClone), Tyrphostins, Gefitinib (Iressa), PTK787 (Novartis), EMD 72000 (Merck), Emodin, Radicinol, Vemurafenib (B-Raf enzyme inhibitor, Daiichi Sankyo), SR-27897 (CCK A inhibitor, Sanofi-Synthelabo), tocladesine (cyclic AMP agonist, Ribapharm), alvocidib (CDK inhibitor, Aventis), CV-247 (COX-2 inhibitor, Ivy Medical), P54 (COX-2 inhibitor, Phytopharm), CapCell™ (CYP450 stimulant, Bavarian Nordic), GCS-100 (ga13 antagonist, GlycoGenesys), G17DT immunogen (gastrin inhibitor, Aphton), efaproxiral (oxygenator, Allos Therapeutics), PI-88 (heparanase inhibitor, Progen), tesmilifene (histamine antagonist, YM BioSciences), histamine (histamine H2 receptor agonist, Maxim), tiazofurin (IMPDH inhibitor, Ribapharm), cilengitide (integrin antagonist, Merck KGaA), SR-31747 (IL-1 antagonist, Sanofi-Synthelabo), CCI-779 (mTOR kinase inhibitor, Wyeth), exisulind (PDE V inhibitor, Cell Pathways), CP-461 (PDE V inhibitor, Cell Pathways), AG-2037 (GART inhibitor, Pfizer), WX-UK1 (plasminogen activator inhibitor, Wilex), PBI-1402 (PMN stimulant, ProMetic LifeSciences), bortezomib (proteasome inhibitor, Millennium), SRL-172 (T cell stimulant, SR Pharma), TLK-286 (glutathione S transferase inhibitor, Telik), PT-100 (growth factor agonist, Point Therapeutics), midostaurin (PKC inhibitor, Novartis), bryostatin-1 (PKC stimulant, GPC Biotech), CDA-II (apoptosis promotor, Everlife), SDX-101 (apoptosis promotor, Salmedix), rituximab (CD20 antibody, Genentech, carmustine, Mitoxantrone, Bleomycin, Absinthin, Chrysophanic acid, Cesium oxides, BRAF inhibitors, PDL1 inhibitors, MEK inhibitors, bevacizumab, angiogenesis inhibitors, dabrafenib, ceflatonin (apoptosis promotor, ChemGenex)

BCX-1777 (PNP inhibitor, BioCryst), ranpirnase (ribonuclease stimulant, Alfacell), galarubicin (RNA synthesis inhibitor, Dong-A), tirapazamine (reducing agent, SRI International), N, acetylcysteine (reducing agent, Zambon), R-flurbiprofen (NF-kappaB inhibitor, Encore), 3CPA (NF-kappaB inhibitor, Active Biotech), seocalcitol (vitamin D receptor agonist, Leo), 131-I-TM-601 (DNA antagonist, TransMolecular), eflornithine (ODC inhibitor, ILEX Oncology), minodronic acid (osteoclast inhibitor, Yamanouchi), indisulam (p53 stimulant, Eisai), aplidine (PPT inhibitor, PharmaMar), gemtuzumab (CD33 antibody, Wyeth Ayerst), PG2 (hematopoiesis enhancer, Pharmagenesis), Immunol™ (triclosan oral rinse, Endo), triacetyluridine (uridine prodrug, Wellstat), SN-4071 (sarcoma agent, Signature BioScience), TransMID-107™ (immunotoxin, KS Biomedix), PCK-3145 (apoptosis promotor, Procyon), doranidazole (apoptosis promotor, Pola), CHS-828 (cytotoxic agent, Leo), trans-retinoic acid (differentiator, NIH), MX6 (apoptosis promotor, MAXIA), apomine (apoptosis promotor, ILEX Oncology), urocidin (apoptosis promotor, Bioniche), Ro-31-7453 (apoptosis promotor, La Roche), brostallicin (apoptosis promotor, Pharmacia), β-lapachone, gelonin, cafestol, kahweol, caffeic acid, Tyrphostin AG, PD-1 inhibitors, CTLA-4 inhibitors, sorafenib, BRAF inhibitors.

In some embodiments of any of the above described methods, the antiproliferative agent is selected from bevacizurnab, bortezomib, capecitabine, cetuximab, fluorouracil, imatinib, irinotecan, leucovorin, oxaliplatin, panitumumab, pemetrexed, temozolomide, cisplatin, paclitaxel, erlotinib, sunitinib, lapatinib, sorafenib, carboplatin, doxorubicin, docetaxel, gemcitabine, etoposide, gefitinib, PD153035, cetuximab, bevacizumab, panitumumab, trastuzumab, anti-c-Met antibodies, gefitinib, ZD6474, EMD-72000, pariitumab, ICR-62, CI-1033, lapatinib, AEE788, EKB-569, EXEL 7647/EXEL 0999, erlotinib, imatinib, sorafinib, sunitinib, dasatinib, vandetinib, temsirolimus, PTK787, pazopanib, AZD2171, everolimus, seliciclib, AMG 706, axitinib, PD0325901, PKC-412, CEP701, XL880, bosutinib, BIBF1120, BIBF1120, nilotinib, AZD6244, HKI-272, MS-275, BI2536, GX15-070, AZD0530, enzastaurin, MLN-518, ARQ197, CM101, IFN-.alpha., IL-12, platelet factor-4, suramin, SU5416, thrombospondin, VEGFR antagonists, angiostatic steroids plus heparin, Cartilage-Derived Angiogenesis Inhibitory Factor, matrix metalloproteinase inhibitors, batimastat, marimastat, angiostatin, endostatin, 2-methoxyestradiol, tecogalan, thrombospondin, .alpha.V.beta.3 inhibitors, linomide, and ADH-1, chlorambucil, cyclophosphamide, ifosfamide, mechlorethamine, melphalan, uracil mustard, thiotepa, busulfan, carmustine, lomustine, streptozocin, carboplatin, cisplatin, satraplatin, oxaliplatin, altretamine, ET-743, XL119, dacarbazine, chlormethine, bendamustine, trofosfamide, uramustine, fotemustine, nimustine, prednimustine, ranimustine, semustine, nedaplatin, triplatin tetranitrate, mannosulfan, treosulfan, temozolomide, carboquone, triaziquone, triethylenemelamine, procarbazin, doxorubicin, daunorubicin, epirubicin, idarubicin, anthracenedione, mitoxantrone, mitomycin C, bleomycin, dactinomycin, plicatomycin, irinotecan, camptothecin, rubitecan, belotecan, etoposide, teniposide, topotecan, paclitaxel, taxol, docetaxel, BMS-275183, xyotax, tocosal, vinorlebine, vincristine, vinblastine, vindesine, vinzolidine, etoposide, teniposide, ixabepilone, larotaxel, ortataxel, tesetaxel, ispinesib, fluorouracil, floxuridine, methotrexate, xeloda, arranon, leucovorin, hydroxyurea, thioguanine, mercaptopurine, cytarabine, pentostatin, fludarabine phosphate, cladribine, asparaginase, gemcitabine, pemetrexed, bortezomib, aminopterin, raltitrexed, clofarabine, enocitabine, sapacitabine, azacitidine.

It will be appreciated by persons skilled in the art that invention described herein are not limited to what has been particularly shown and described. Rather, the scope of the invention is defined by the claims which follow. It should further be understood that the above description is only representative of illustrative examples of embodiments. The description has not attempted to exhaustively enumerate all possible variations. The alternate embodiments may not have been presented for a specific component of the drug combination, or a step of the method, and may result from a different combination of described constituents, or that other un-described alternate embodiments may be available for a combination or method, is not to be considered a disclaimer of those alternate embodiments. It will be appreciated that many of those un-described embodiments are within the literal scope of the following claims, and others are equivalent.

1. A drug combination for treating a neoplasm in a subject, comprising:
poziotinib or a pharmaceutically acceptable salt thereof; and
a vascular endothelial growth factor receptor 2 (VEGFR2) inhibitor.

2. The drug combination of claim 1, wherein the VEGFR2 inhibitor is Ramucirumab.

3. The drug combination of claim 1, wherein the poziotinib is in a monohydrochloride salt form.

4. A kit for treating a neoplasm in a subject, comprising:
poziotinib or a pharmaceutically acceptable salt thereof; and
a vascular endothelial growth factor receptor 2 (VEGFR2) inhibitor.

5. The kit of claim 4, wherein the VEGFR2 inhibitor is Ramucirumab.

6. The kit of claim 4, further comprising an educational information and direction of use.

7. A method for treating a neoplasm in a subject, comprising administering a drug combination according to claim 1.

8. The method of claim 7, wherein the neoplasm is selected from the group consisting of non-small cell lung cancer, breast cancer, stomach cancer, colon cancer, pancreatic cancer, prostate cancer, myeloma, head and neck cancer, ovarian cancer, esophageal cancer, and metastatic cell carcinoma.

9. The method of claim 7, wherein the VEGFR2 inhibitor in the drug combination is Ramucirumab.

10. The method of claim 7, wherein the subject expresses a mutation at exon 18, exon 19, exon 20, or exon 21.

11. The method of claim 7, further comprising determining the subject as having one or more HER2 exon 20 mutations.

12. The method of claim 7, further comprising determining the subject as having one or more EGFR exon 20 mutations selected from the group consisting of A775insV G776C, A775insYVMA, G776C V777insC, G776del insVV, G776del insVC, P780insGSP, Y772dupYVMA, V773M, G776delinsLC, V777L, V777insCG, G778dupGSP, P780insGSP, L786V.

13. The method of claim 7, further comprising determining the subject as having one or more EGFR exon 20 mutations selected from the group consisting of A763insFQEA, A767insASV, S768dupSVD, V769insASV, D770insSVD, D770insNPG, H773insNPH, N771del insGY, N771del insFH, N771dupNPH, A767insTLA, S768I, V769L, V769insGSV, D770del insGY, D770insG, D770insY H773Y, N771insHH, P772insDNP, H773insAH, H773insH, V774insHV, S784F, R776C, V774M, V769M, G796D, S784F, C775Y, S811F, T790M, V774A, D770A.

14. The method of claim 7, further comprising determining the subject as having one or more HER2 exon 21 mutations selected from the group consisting of a point mutation, insertion, and deletion of 1-18 nucleotides between amino acids 832-883.

15. The method of claim 7, further comprising determining the subject as having one or more HER2 exon 21 mutations are at one or more residues selected from the group consisting of V842, R868, and L869.

16. The method of claim 7, wherein the neoplasm is non-small cell lung carcinoma.

17. The method of claim 7, wherein the neoplasm is a metastatic cancer.

18. The method of claim 7, wherein the cancer is refractory to chemotherapy or radiotherapy, resistant to chemotherapy, or has relapsed.

19. The method of claim 7, wherein the subject has previously received one, two, three or more lines of therapy for the neoplasm.

20. The method of claim 7, wherein the subject has not previously received treatment with a EGFR tyrosine kinase inhibitor.

21. The method of claim 7, wherein the subject has received prior treatment with an EGFR tyrosine kinase inhibitor.

22. The method of claim 7, wherein the administration and/or dosage of the drug combination is controlled to treat or prevent CNS metastases in the subject.

23. A method of treating or preventing CNS metastases in a subject, wherein the subject has been diagnosed to have a cancer comprising administering a drug combination according to claim 1.

24. The method of claim 23, wherein the cancer is non-small cell lung cancer (NSCLC).

25. The method of claim 23, wherein the subject has been determined to have CNS metastases.

26. The method of claim 23, wherein the subject has been determined to have no CNS metastases.

27. The method of claim 23, wherein the subject has previously received one, two, three or more lines of therapy for the neoplasm.

28. The method of claim 23, wherein the subject has not previously received treatment with a EGFR tyrosine kinase inhibitor.
